(19) United States
(12) Patent Application Publication
(10) Pub. No.: US 2026/0215713 A1
(43) Pub. Date: Jul. 30, 2026

(54) Method For Calculating A Stress Tolerance Rating Of A Fighter Pilot

(71) Applicant: Airbus Defence and Space GmbH, Taufkirchen (DE)

(72) Inventors: Konrad Auenhammer, Taufkirchen (DE); Stephen Horn, Taufkrichen (DE); Martin Knaup, Taufkirchen (DE)

(73) Assignee: Airbus Defence and Space GmbH, Taufkirchen (DE)

(21) Appl. No.: 19/457,589

(22) Filed: Jan. 23, 2026

(30) Foreign Application Priority Data
Jan. 24, 2025 (DE) 102025000300.5

(51) Int. Cl. A61B 5/18; A61B 5/00; A61B 5/08; A61B 5/16; A62B 7/14; A62B 9/00; B64D 10/00

(52) U.S. Cl. CPC A61B 5/18; A61B 5/082; A61B 5/165; A61B 5/6803; B64D 10/00; A61B 2503/22; A61B 2562/0219; A62B 7/14; A62B 9/00

(57) ABSTRACT

The method uses a nitrogen monoxide (NO) sensor arranged in an exhalation channel of a breathing mask, which includes a pressure ventilation device, an acceleration sensor, a ventilation pressure sensor arranged in a breathing air channel of the breathing mask. A computer calculates an individual, unadjusted stress tolerance rating and uses the measured value of the NO sensor together with historical measured values and associated, historical calculation values, and calculates out the influences of the acceleration and the pressure ventilation acting on the fighter pilot and uses measured values of the acceleration sensor and of the ventilation pressure sensor together with historical measured values of the acceleration sensor and of the ventilation pressure sensor and associated historical calculation values while obtaining an individualized, adjusted stress tolerance rating. The historical measured values and the historical calculation values are stored in the memory and are data of the fighter pilot.

### CROSS REFERENCE TO RELATED APPLICATIONS

The present application claims priority from German Application No. 102025000300.5 filed Jan. 24, 2025, the disclosure of which is hereby incorporated herein by reference.

The invention relates to a method for calculating an individualized, stress tolerance rating of a fighter pilot, wherein a biomarker sensor is used.

The term “measured value” is to be interpreted broadly and can comprise individual values, time-resolved values, integral values or differential values. Likewise, the term “calculation value” is to be interpreted broadly, since it relates to a measured value.

U.S. Pat. No. 8,892,274 B2 shows a method for calculating an individualized stress tolerance rating of a helicopter pilot for a helicopter assistance system. The method uses exclusively physiological sensors, such as, for example, a brain activity sensor, a respiratory rate sensor or a temperature sensor. The method further uses an evaluation device with a computer and a memory. The computer processes with a computer program the data of the sensors together with historical data of the helicopter pilot and calculates an individualized stress tolerance rating. The individualized stress tolerance rating controls an assistance system. A high stress tolerance rating requires a high level of support by the assistance system.

US 2021/0015399 A1 shows a biomarker sensor, with which the content of the biomarker nitrogen monoxide (NO) in combination with the biomarker isoprene can be detected. The biomarker sensor can be used to generate a hypoxia warning value for fighter pilots who are subjected to high accelerations.

In one of the numerous exemplary embodiments, U.S. Pat. No. 11,839,781 B1 shows, for example, a method for calculating an individualized hypoxia warning value of a fighter pilot. The method can be configured differently by selecting individual modules, as illustrated in FIG. 17 of U.S. Pat. No. 11,839,781 B1. In addition to many other sensors, an acceleration sensor and a ventilation pressure sensor, which is arranged in a breathing air channel of a breathing mask, can also be used. Although a wide variety of sensors are mentioned, an NO sensor, a biomarker sensor, is not mentioned. The method further uses a configurable evaluation device with a computer and a memory for storing historical data. The computer processes with a computer program data of the sensors together with historical data of the fighter pilot, in order, for example, to determine an individualized hypoxia warning value.

U.S. Pat. No. 12,085,507 B2 shows a sensor arrangement with a CO2 sensor and an H2O sensor for a method for calculating a hypoxia warning value. The sensor arrangement operates according to the principle of infrared spectroscopy. The sensor arrangement is arranged in an exhalation channel of the exhaled air of a breathing mask. A computer processes with a computer program the data of the sensors together with historical data of the fighter pilot and calculates a hypoxia warning value.

U.S. Pat. No. 10,595,758 B2 shows a hypoxia sensor which is a pulse oximeter.

The invention is based on the object of developing, starting from U.S. Pat. No. 8,892,274 B2, an alternative method for calculating a stress tolerance rating of a pilot. Further objects are the development of a corresponding computer program, a corresponding computer-readable medium and a corresponding device.

These objects are achieved according to the invention by claim 1 directed to a method, by claim 7 directed to a computer program, by claim 8 directed to a computer-readable medium and by claim 9 directed to a device.

The advantages of the invention are that a meaningful stress tolerance rating which is tailored exactly to fighter pilots is determined in a simple but reliable manner. A stress tolerance rating can be used to control an assistance system. If a stress tolerance rating falls, the assistance system has to take more tasks. The core idea of how a meaningful stress tolerance rating for fighter pilots is determined is that firstly a biomarker sensor, an NO sensor, is used which measures the content of NO. An unadjusted stress tolerance rating is derived therefrom. An adjustment takes place by a measured value being detected with an acceleration sensor, which measured value represents the influence of the acceleration acting on the fighter pilot, which acceleration represents a first environmental parameter to be factored out. A further adjustment takes place by the ventilation pressure sensor detecting a measured value, which represents the influence of the pressure ventilation acting on the fighter pilot, which pressure ventilation represents a second environmental parameter to be factored out. Historical data of the pilot are used for individualization.

Once again referring to the prior art presented above, U.S. Pat. No. 8,892,274 B2 does not comprise a biomarker in the method for calculating an individualized stress tolerance rating of a helicopter pilot for a helicopter assistance system. Another document, U.S. Pat. No. 2021/0015399 A1, admittedly proposes using an NO sensor, which is a biomarker sensor, in order to generate a hypoxia warning. However, no unadjusted stress tolerance rating is derived with the NO sensor, in which the influence of the acceleration acting on the fighter pilot and the influence of the pressure ventilation acting on the fighter pilot are factored out in order to obtain an individualized, adjusted stress tolerance rating.

An advantageous embodiment of the invention reproduces the features of claim 2. A simple formula is described which is comprised by the calculation steps of calculating an individualized, adjusted stress tolerance rating.

A further advantageous embodiment of the invention reproduces the features of claim 3. According to this, the NO sensor can be used as the sole biomarker sensor. Likewise, a second biomarker sensor can be used in combination with the NO sensor. The second biomarker is selected from a group which represents suitable biomarker sensors.

A further advantageous embodiment of the invention is shown by the features of claim 4. The temperature of the exhaled breath leads to the body temperature. The temperature sensor arranged in the exhalation channel 3 detects a measured value, which represents the influence of the body temperature of the fighter pilot, which influence is likewise to be factored out.

Further advantageous embodiments of the invention are shown by the self-explanatory features of claim 5 and of claim 6.

Exemplary embodiments of the invention are described in more detail with reference to the drawings. In the figures:

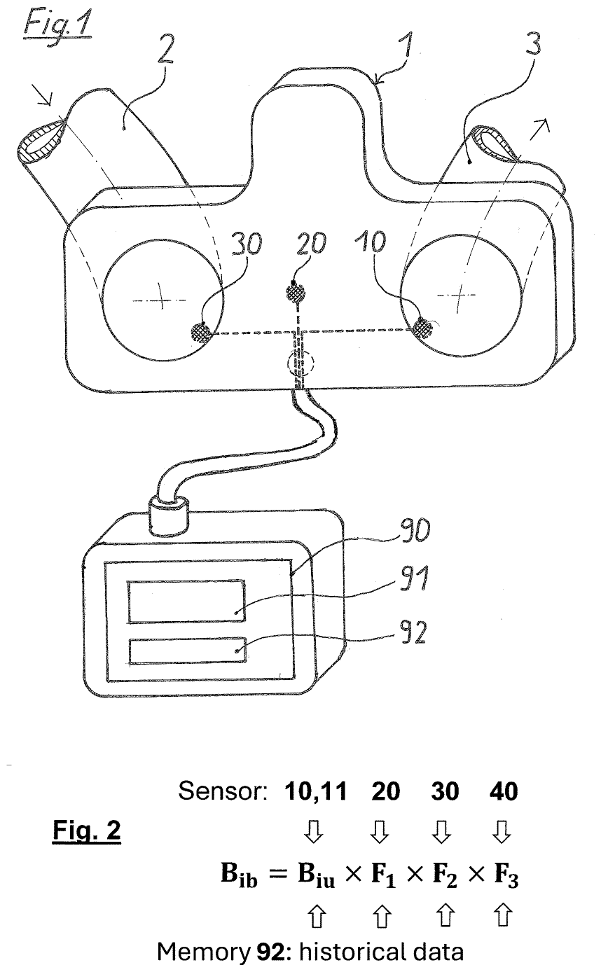

FIG. 1 shows a breathing mask, sensors which are attached to the breathing mask, and an evaluation device, as a perspective schematic diagram,

FIG. 2 shows a formula for calculating an individualized, adjusted stress tolerance rating of a fighter pilot.

FIG. 1 illustrates a first exemplary embodiment, in which an individualized, adjusted stress tolerance rating B_{ib} of a fighter pilot is calculated using a biomarker sensor and two environmental parameter sensors. The method uses, as the sole biomarker sensor, an NO sensor 10, which is arranged in an exhalation channel 3 of a breathing mask 1, which comprises a pressure ventilation device. For miniaturization, the NO sensor 10 comprises a ChemFET (chemical field-effect transistor). A ChemFET is a special type of field-effect transistor which is used for detecting chemical substances. It functions by converting changes in the chemical environment, such as the presence of certain molecules, into electrical signals.

The breathing mask 1 is attached to a helmet (not shown). The method further uses an acceleration sensor 20, which is arranged, for example, on the breathing mask 1 and records translational and rotational accelerations in all three spatial directions. The method uses a ventilation pressure sensor 30, which is arranged in a breathing air channel 2 of the breathing mask 1. The ventilation pressure sensor 30 operates non-intrusively according to the Doppler principle or according to the calorimetric principle. The acceleration sensor 20 and the ventilation pressure sensor 30 comprise MEMS (micro-electro-mechanical systems). MEMS are miniaturized systems which combine mechanical and electronic components on a single chip. Finally, the method uses an evaluation device 90 with a computer 91, a memory 92 and a real-time clock (RTC) device in order to provide the measured values of the sensors 10, 20, 30 with a time stamp.

The steps of the method are:
a) the method uses an NO sensor 10, which is arranged in an exhalation channel 3 of a breathing mask 1, which comprises a pressure ventilation device, an acceleration sensor 20, a ventilation pressure sensor 30, which is arranged in a breathing air channel 2 of the breathing mask 1, a computer 91 and a memory 92, and comprises the following steps:
b) the NO sensor 10, a biomarker sensor, detects a measured value of the NO content in the exhalation air of the fighter pilot,
c) the acceleration sensor 20 detects a measured value, which represents the influence of the acceleration acting on the fighter pilot,
d) the ventilation pressure sensor 30 detects a measured value, which represents the influence of the pressure ventilation acting on the fighter pilot, wherein it is noted that a breathing rate can also be determined, for example, with the ventilation pressure sensor 30,
e) the computer calculates an individual, unadjusted stress tolerance rating Bᵢᵤ and uses for this purpose the measured value of the NO sensor 10 together with historical measured values and associated, historical calculation values,
f) the computer calculates out the influence of the acceleration acting on the fighter pilot and the influence of the pressure ventilation acting on the fighter pilot and uses for this purpose measured values of the acceleration sensor 20 and of the ventilation pressure sensor 30 together with historical measured values of the acceleration sensor 20 and of the ventilation pressure sensor 30 and associated historical calculation values while obtaining an individualized, adjusted stress tolerance rating B_{ib},
g) wherein the historical measured values and the historical calculation values are stored in the memory and are data of the fighter pilot.

The individualized, adjusted stress tolerance rating B_{ib} is calculated with calculation steps which comprise the following formula:

$${B}_{ib} = {B}_{iu} \times {F}_{1} \times {F}_{2}$$

wherein
   Bᵢᵤ is an individualized, unadjusted stress tolerance rating, calculated as the function value of the function ƒ(NO), wherein ƒ(NO) are historical calculation values of historical measured values of the NO sensor 10,
   F₁ is a first adjustment factor, calculated as the function value of the function ƒ(acceleration), wherein ƒ(acceleration) are historical calculation values of historical measured values of the acceleration sensor 20,
   F₂ is a second adjustment factor, calculated as the function value of the function ƒ(ventilation pressure), wherein ƒ(ventilation pressure) are historical calculation values of historical measured values of the ventilation pressure sensor 30.

The method operates autonomously and uses independent sensors 10, 11, 20, 30, 40, which are independent of avionics of an aircraft of the fighter pilot.

The individualized, adjusted stress tolerance rating B_{ib} is used in an assistance system of a fighter aircraft of the fighter pilot and controls the degree of assistance.

The computer program comprises commands which cause the computer 91 to carry out the method steps. The computer program is stored on the internal memory of the computer 91 and as a backup on a computer-readable medium.

A second exemplary embodiment is described below with reference to a supplemented formula which is shown in FIG. 2. Only the differences with respect to the first exemplary embodiment are presented.

An unadjusted stress tolerance rating Bᵢᵤ is now calculated with an NO sensor 10 in combination with an isoprene sensor 11, a further biomarker sensor.

The method also uses a temperature sensor 40, which is arranged in the exhalation channel 3. The temperature sensor 40 detects a measured value, which represents the influence of the body temperature. This influence is factored out by the computer 91 using measured values of the temperature sensor 40 and associated historical calculation values. F₃ is a third adjustment factor, which is calculated as the function value of the function ƒ(temperature), wherein ƒ(temperature) are historical calculation values of historical measured values of the temperature sensor 40.

In contrast to the first and second exemplary embodiments, the following modifications are possible, for example:
The second biomarker sensor in the second exemplary embodiment can also be an estradiol sensor, a 2-methylpentadecane sensor, an indole sensor, a benzaldehyde sensor, a 2-hydroxy-1-phenylethan-1-one sensor or a 2-ethylhexanol sensor.
The method can additionally use a moisture sensor, which is arranged in the exhalation channel 3. The moisture sensor detects a measured value, which represents the influence of the dehydration acting on the fighter pilot. This influence is factored out by the computer using measured values of the moisture sensor and associated historical calculation values. A decreasing air humidity offers a conclusion about a possible dehydration associated with a mental performance drop.
In the first and second exemplary embodiments, the method uses a ventilation pressure sensor 30, which is arranged in a breathing air channel 2 of the breathing mask 1. In addition, an exhalation pressure sensor, which is arranged in the exhalation channel 3, can be used. The breathing characteristic can be detected with an additional exhalation pressure sensor in order to more accurately determine the influence of the pressure ventilation on the fighter pilot.

### REFERENCE SIGNS

1 Breathing mask
2 Breathing air channel
3 Exhalation channel
10 NO sensor
11 Isoprene sensor
20 Acceleration sensor
30 Ventilation pressure sensor
40 Temperature sensor
90 Evaluation device
91 Computer
92 Memory

1. A method for calculating an individualized, adjusted stress tolerance rating (B_{ib}) of a fighter pilot, comprising:
a) using a nitrogen monoxide (NO) sensor arranged in an exhalation channel of a breathing mask, the breathing mask comprising a pressure ventilation device, an acceleration sensor, a ventilation pressure sensor arranged in a breathing air channel of the breathing mask, a computer and a memory;
b) detecting via the NO sensor, a biomarker sensor, a measured value of the NO content in the exhalation air of the fighter pilot,
c) detecting via the acceleration sensor a measured value representing an influence of the acceleration acting on the fighter pilot,
d) detecting via the ventilation pressure sensor a measured value representing an influence of the pressure ventilation acting on the fighter pilot,
e) calculating by the computer an individual, unadjusted stress tolerance rating (Bᵢᵤ) using the measured value of the NO sensor together with historical measured values and associated, historical calculation values, and
f) calculating by the computer the influence of the acceleration acting on the fighter pilot and the influence of the pressure ventilation acting on the fighter pilot using the measured values of the acceleration sensor and of the ventilation pressure sensor together with historical measured values of the acceleration sensor and of the ventilation pressure sensor and associated historical calculation values while obtaining an individualized, adjusted stress tolerance rating (B_{ib}),
g) wherein the historical measured values and the historical calculation values are stored in the memory and are data of the fighter pilot.

2. The method according to claim 1, in which the individualized, adjusted stress tolerance rating (B_{ib}) is calculated with calculation steps which comprise the following formula:
$${B}_{ib} = {B}_{iu} \times {F}_{1} \times {F}_{2}$$
wherein
Bᵢᵤ is an individualized, unadjusted stress tolerance rating, calculated as the function value of the function ƒ(NO), wherein ƒ(NO) are historical calculation values of historical measured values of the NO sensor,
F₁ is a first adjustment factor, calculated as the function value of the function ƒ(acceleration), wherein ƒ(acceleration) are historical calculation values of historical measured values of the acceleration sensor,
F₂ is a second adjustment factor, calculated as the function value of the function ƒ(ventilation pressure), wherein ƒ(ventilation pressure) are historical calculation values of historical measured values of the ventilation pressure sensor.

3. The method according to claim 1, wherein, for calculating an individualized, unadjusted stress tolerance rating (Bᵢᵤ), the NO sensor is used either alone or in combination with a further biomarker sensor from a group consisting of an isoprene sensor, an estradiol sensor, a 2-methylpentadecane sensor, an indole sensor, a benzaldehyde sensor, a 2-hydroxy-1-phenylethan-1-one sensor, and a 2-ethylhexanol sensor.

4. The method according to claim 1, further comprising using a temperature sensor arranged in the exhalation channel, wherein the temperature sensor detects a measured value representing an influence of the body temperature of the fighter pilot and wherein the influence of the body temperature is factored out by the computer using measured values of the temperature sensor and associated historical calculation values.

5. The method according to claim 1, further comprising using independent sensors which are independent of avionics of an aircraft of the fighter pilot.

6. The method according to claim 1, further comprising using the individualized, adjusted stress tolerance rating (B_{ib}) in an assistance system of a fighter aircraft of the fighter pilot.

7. A non-transitory computer-readable medium comprising a computer program configured to, when executed, to cause the computer to carry out the method according to claim 1.

8. A device for calculating an individualized, adjusted stress tolerance rating (B_{ib}) of a fighter pilot, comprising:
a) an NO sensor arranged in an exhalation channel of a breathing mask, the breathing mask comprising a pressure ventilation device, an acceleration sensor, a ventilation pressure sensor arranged in a breathing air channel of the breathing mask, a computer and a memory,
b) wherein the device is configured to carry out the method according to claim 1.
